(19) United States
(12) Patent Application Publication
(10) Pub. No.: US 2007/0006876 A1
(43) Pub. Date: Jan. 11, 2007

(54) Add-on spacer design concept for dry-powder inhalers

(75) Inventors: Warren H. Finlay, Edmonton (CA); Edgar A. Matida, Edmonton (CA); Carlos F. Lange, Edmonton (CA)

Correspondence Address: OGILVY RENAULT LLP, 1981 MCGILL COLLEGE AVENUE, SUITE 1600, MONTREAL, QC, H3A2Y3, CA

(73) Assignee: University of Alberta, Alberta (CA)

(21) Appl. No.: 10/556,048

(22) PCT Filed: May 14, 2004
(86) PCT No.: PCT/CA04/00737

§ 371 Date: Nov. 8, 2005

Related U.S. Application Data
(60) Provisional application No. 60/470,852, filed on May 16, 2003.

(51) Int. Cl. A61M 11/00

(52) U.S. Cl. 128/200.14

(57) ABSTRACT

A spacer, for disposition between a user's mouth and a medicament inhaler outlet, has a hollow body defining an elongate internal chamber (10) with a diffuser portion (8) having a spacer inlet (9) adapted to engage the inhaler outlet in communication with the internal chamber, the diffuser portion extending axially outwardly from the spacer inlet; a buffer portion (6) extending axially from the diffuser portion; and a nozzle portion (7) having a spacer outlet (5) adapted to engage the user's mouth in communication with the internal chamber, the nozzle portion extending axially inwardly from the buffer portion.

### TECHNICAL FIELD

The invention relates to an add-on spacer design concept for a commercial dry powder inhaler that enhances total aerosol medication dose delivery distal to a patient's mouth-throat area.

### BACKGROUND OF THE ART

Nebulizers, MDIs (pressurized metered dose inhalers) and DPIs (dry powder inhalers) are devices used to generate medication in the form of solid or liquid particles, which are often inhaled by patients in the treatment of lung diseases, such as asthma and bronchitis. Although the lung is the final target, part of the dose will deposit on the walls of the extrathoracic region, from the mouth opening to the end of the trachea, resulting in dosage losses and departure from the ideal delivery. Waste of medication and any unpredictable variation in dosage delivered are clearly undesirable.

Mouth-throat deposition of inhaled pharmaceutical aerosols is normally undesirable since the intended target is the lungs. Unintentional deposition of active pharmaceutical ingredients (API) in the mouth-throat can result in local side effects (e.g. candidiasis, dysphonia, dry mouth, infection) as well as unintended ingestion of API into the gastrointestinal tract that can result in systemic side effects (e.g. adrenal suppression and stunted growth, osteoporosis, immunosuppression, fluid retention, weight gain, mood swings, dermal thinning, cataracts, muscle weakness). For these reasons, reducing mouth-throat deposition with inhaled aerosol delivery is desirable.

Experimental results have shown that the aerosol deposition efficiency in the extrathoracic region of human subjects is a function of a simplified inertial parameter, ρₚ dₚ²Q, where ρₚ is the particle density, dₚ is the particle diameter and Q is the inhalation flow rate. The complete inertial parameter is ρₚ dₚ²Q/18 μL including the viscosity of gas μ and characteristic length scale of the fluid flow path L. However since the viscosity of breathable gas does not vary significantly, the simplified parameter is more commonly used. Add-on devices, called spacers or mouthpieces, are conventionally used to enhance the original performance of the inhalers when attached to them, by increasing the total amount of drug delivered into the lungs from the same dosage. Although studies are more established with MDIs, only a relatively few studies concerning add-on devices for DPIs have appeared in the literature.

DPIs (Dry Powder Inhalers) were the latest of the three types of inhalation devices to be developed, mainly due to difficulties associated with powder manufacturing, agglomeration and dispersion issues. Dry powder particles have the tendency to adhere to each other and any surrounding surfaces. DPI devices try to deagglomerate adhered particles using impaction, entrainment mechanisms, swirling flows, grid turbulence, jets and impinging jets. The size distribution of deagglomerated particles is a function of inhalation flow rates. The peak inspiratory flow rates depend on the patients and the DPI design. DPIs have normally a relatively small outlet diameter, usually up to 10 mm diameter. Experimental analysis of two commercial DPIs has shown undesirably high deposition losses in an idealized extrathoracic cast. The prior art demonstrates by aerosol deposition measurements in the mouth cavity, that an increase in the inlet diameter, from 3 mm to 17 mm, can decrease aerosol deposition in the mouth cavity, by reducing the influence of the inhaler jet impingement on the posterior wall of the mouth. In the present invention, a spacer design is provided, bringing the highly turbulent and complex flow from a Turbuhaler* DPI device to an outlet of lower velocities and lower turbulence intensities, having at the same time low particle deposition rates in the spacer itself, giving less particle deposition in the mouth cavity and enhancing particle delivery through the mouth-throat region.
*Trade-mark of Astra Pharma Inc.

Further objects of the invention will be apparent from review of the disclosure, drawings and description of the invention below.

### DISCLOSURE OF THE INVENTION

The invention provides a spacer, for disposition between a user's mouth and a medicament inhaler outlet, has a hollow body defining an elongated internal chamber with a diffuser portion having a spacer inlet adapted to engage the inhaler outlet in communication with the internal chamber, the diffuser portion extending axially outwardly from the spacer inlet; a buffer portion extending axially from the diffuser portion; and a nozzle portion having a spacer outlet adapted to engage the user's mouth in communication with the internal chamber, the nozzle portion extending axially inwardly from the buffer portion.

### DESCRIPTION OF THE DRAWINGS

In order that the invention may be readily understood, embodiments of the invention are illustrated by way of example in the accompanying drawings.

FIG. 1(a) shows a simple straight diffuser used as a comparison in experiments described herein.

FIG. 1(b) shows an embodiment of the invention in a diffuser having a diffuser section, a buffer section and a nozzle section, also used in experiments described herein.

FIG. 2 is a diagram of the diffuser of FIG. 1(b) with CFD mesh (Computational Fluid Dynamics) applied at the mid-plane of the spacer geometry.

FIG. 3(a) is an exploded perspective view of the experimental aerosol setup with idealized mouth-throat cast, the spacer in accordance with the invention, the inhaler device and a filter to capture particles delivered.

FIG. 3(b) is a schematic view of the monodisperse aerosol setup used in experiments described herein.

FIG. 4 is a gray scale graphical display of the results of simulations described herein, showing at the left the range in magnitude of fluid mean velocities (speed) and at the right the range of magnitude of turbulent kinetic energy (tke), comparing three configurations of spacers (small, medium and large from top to bottom) in accord with the invention.

FIG. 5 is a bar graph display of experimental results comparing the percent of medication by weight deposited at different locations along the path that medication travels downstream, namely through the spacer, the mouth-throat cast and finally caught in the filter, for three configurations namely, no add-on spacer, a simple straight diffuser (as in FIG. 1 (a)) and a spacer according to the invention (as in FIG. 1 (b)).

FIG. 6 is a line graph comparing the simplified inertial parameter ρₚ dₚ²Q for Stahlhofen's curve, with a spacer according to the invention and without a spacer.

FIG. 7 is a view of a conventional Turbuhaler™ DPI device disposed within a spacer according to the invention, to indicate the relative sizes, possible packaging and carrying convenience for use by patients.

FIG. 8 is an axial sectional view of the spacer according to the invention, with interior and exterior boundary envelopes shown in dashed outline to indicate the range of dimensions within which optimal medication delivery is likely to occur depending on the DPI used and patient's characteristics.

Further details of the invention and its advantages will be apparent from the detailed description included below.

### DETAILED DESCRIPTION OF PREFERRED EMBODIMENTS

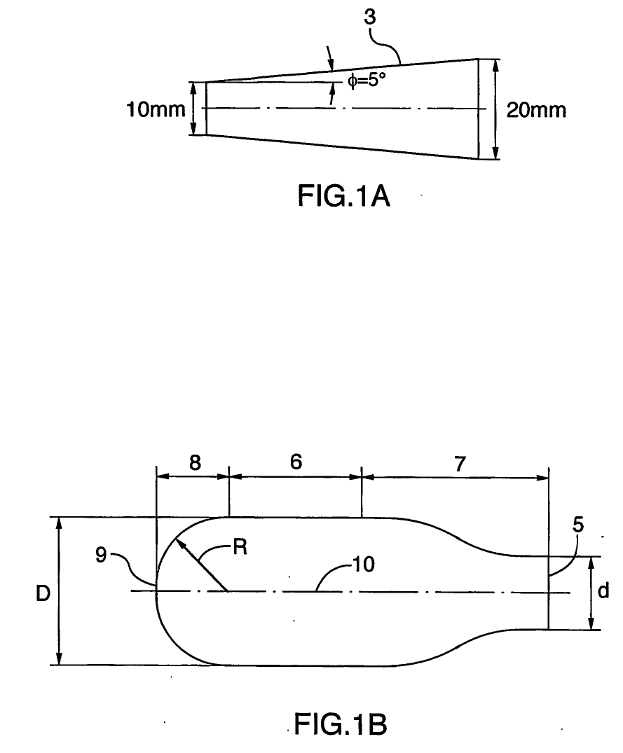

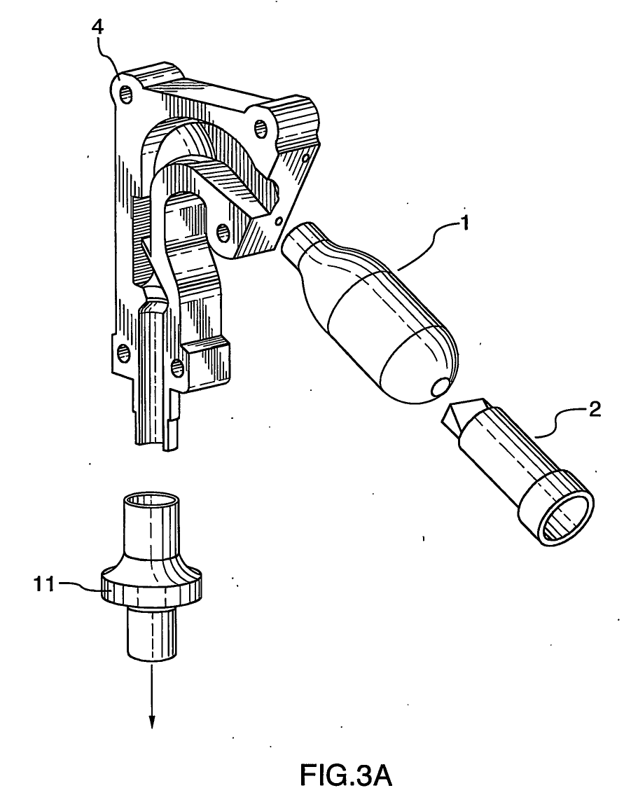

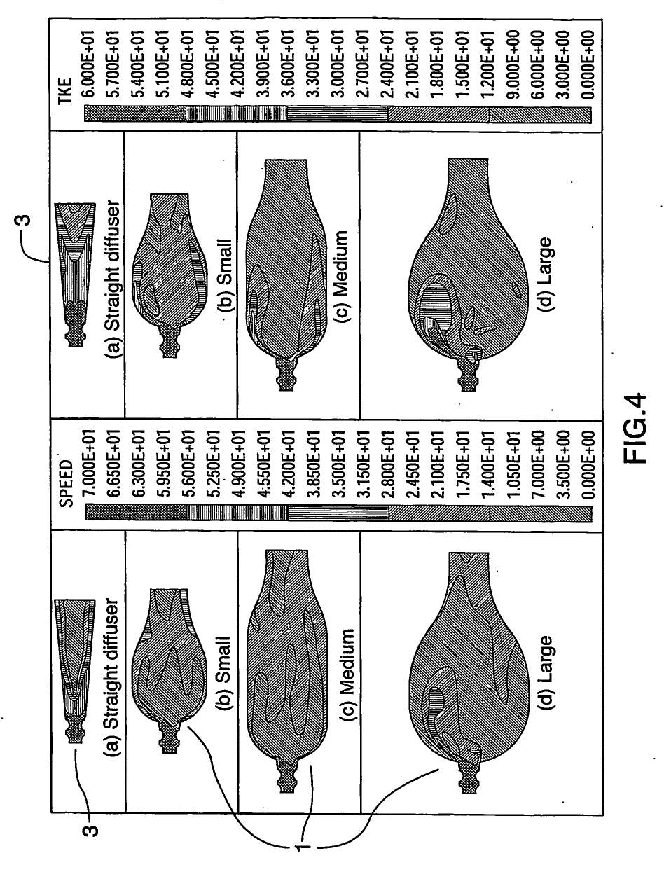

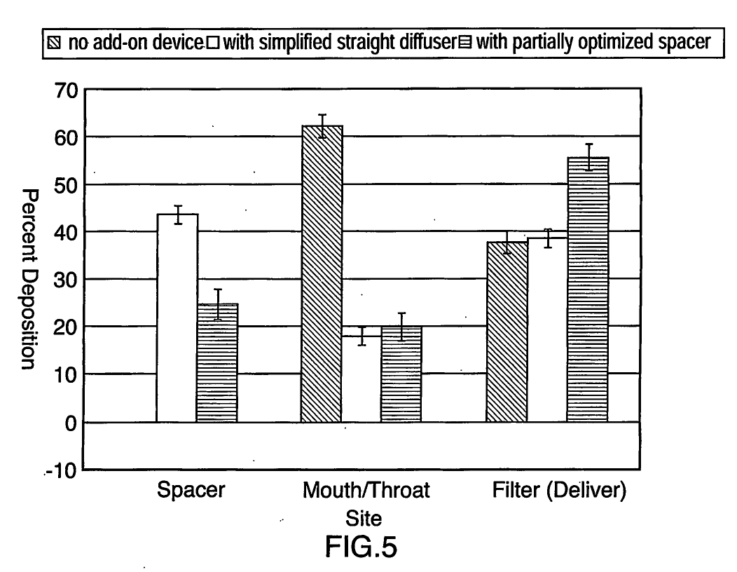

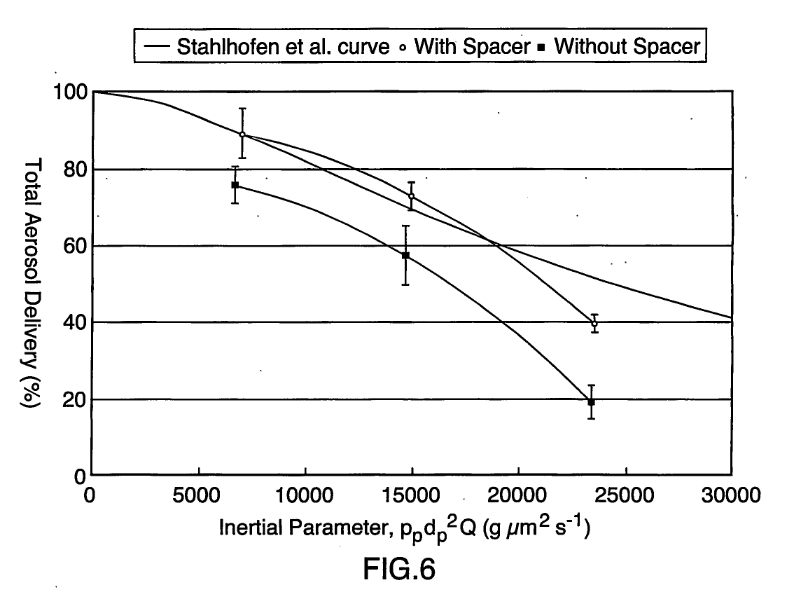

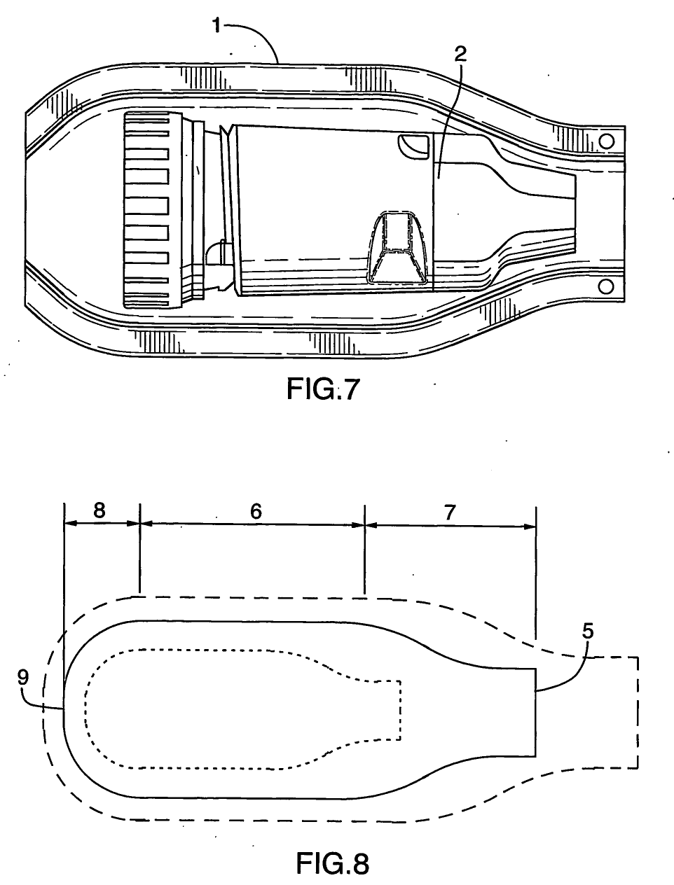

An add-on spacer 1 is provided for use with conventional commercial dry powder inhalers such as a Turbuhaler 2 for example, shown in FIG. 7. In developing the spacer 1 and testing the efficacy, initial design of the spacer 1 was performed using Computational Fluid Dynamics. The performance of a simplified straight diffuser 3 (as shown in FIG. 1(a)) and a spacer 1 with geometry in accordance with the invention (FIG. 1(b)) were evaluated by measuring the impact of these two add-on devices 1, 3 on efficiencies of aerosol deposition in an idealized mouth-throat cast 4 (shown in FIGS. 3(a)-(b)). HPLC and UV spectroscopy techniques (known to those skilled in the art) are used to provide the aerosol deposition measurements illustrated in FIGS. 5-6. Turbuhaler™ (terbutaline sulfate, 500 μg) aerosol and an inhalation flow rate of 70 L/min were used for both the simplified straight diffuser 3 (FIG. 1(a)) and the add-on spacer 1 according to the invention (FIG. 1(b)). In addition, monodisperse aerosols for three simplified inertial parameter values, ρₚ dₚ²Q (g μm² s⁻¹), of 6,635, 14,700 and 23,430 are used for the geometry of the spacer 1 according to the invention.

Compared to Turbuhaler™ alone, in experimental results shown in FIG. 5, the newly developed spacer 1 significantly improved total aerosol delivery distal to the mouth-throat cast by approximately 47% for Turbuhaler aerosol and reduced the deposition in the mouth-throat region. Further in experimental results shown in FIG. 6, the newly developed spacer 1 significantly improved total aerosol delivery distal to the mouth-throat cast 4 by approximately 17, 27 and 107%, respectively, for the above monodisperse aerosols. In contrast, the straight diffuser 3 of FIG. 1(a) did not significantly improve delivery distal to the mouth-throat replica 4.

As stated above, many commercial DPIs (Dry Powder Inhalers) have outlet jets with relatively small diameter (up to 10 mm, and estimated within the range from 7.5 to 12.5 mm) including complex flows, such as swirling jets or converging multiple jets. In the prior art, aerosol deposition measurements in the mouth cavity indicate that the ideal spacer should bring the small outlets of commercial devices to a larger diameter approximately, 20 mm, that is large enough to decrease aerosol deposition in the mouth by decreasing the impinging jet effect, but small enough to accommodate differences in maximum mouth opening of patients.

The simplest method of increasing the outlet size is to provide an add-on straight diffuser device 3 for example with a length of 70 mm smoothly connecting the inhaler device of inlet 10 mm to an outlet with 20 mm diameter, as shown in FIG. 1(a). Following performance guidelines for diffusers which efficiently convert inlet dynamic pressure into static pressure rise by means of a gradual increase in the cross sectional area, trying to avoid separation and stall zones, a divergence half angle of Φ=5° was adopted in the experiments.

Extensive testing of prototypes and numerical simulations led to the spacer 1 of FIG. 1(b). Besides an outlet 5 of approximately 20 mm, and preferably in the range from 15 to 25 mm, it was determined that the spacer 1 should optimally include the features illustrated in FIG. 1(b) with a buffer region 6 in order to allow dissipation of the jets coming from the DPI devices and avoiding aerosol deposition on the interior of the spacer 1 at the same time. A mouthpiece outlet 5 with low turbulence intensities is also desirable, since high turbulence means higher diffusion of aerosol particles, which will convect particles towards adjacent walls. This can be achieved by connecting the buffer region 6 and the outlet 5 with a nozzle 7 as in FIG. 1(b). The nozzle design follows the guidelines for wind tunnel contractions with an ogee curvature. Both designs shown in FIG. 1 are compared in the Turbuhaler aerosol deposition experiment described below.

The spacer 1 therefore includes a diffuser portion 8 having a spacer inlet 9 adapted to engage the inhaler outlet (not shown) in communication with the internal chamber 10 of the spacer 1. The diffuser portion 8 may have a hemi-spherical shape as illustrated with radius R in the range of 15-25 mm which extends axially outwardly from the spacer inlet opening 9 of diameter in the range of 7.5-12.5 mm. The buffer portion 6 extends axially from the diffuser portion 8 and may be a cylindrical shape as illustrated with a diameter D in the range from 30-50 mm and length L_{B} in the range from 30-50 mm. The nozzle portion 7 has a spacer outlet 5 adapted to engage the user's mouth (not shown) in communication with the internal chamber 10. The nozzle portion 7 extends axially inwardly from the buffer portion in an ogee curvature or transition. The nozzle portion 7 may have an upstream diameter D in the range from 30-50 mm, a downstream diameter “d” in the range from 15-25 mm, and a length L_{N} in the range from 37.5-62.5 mm.

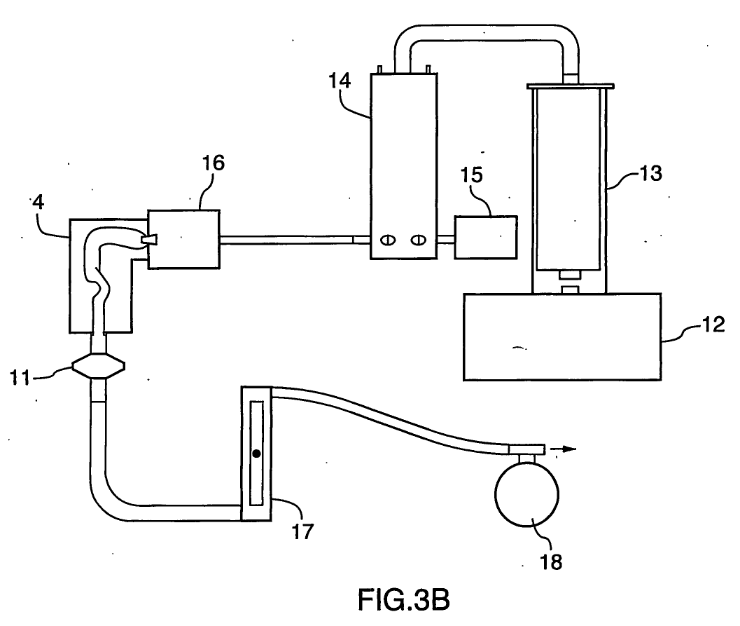

In order to initially select the general dimensions of the spacer 1, optimization was performed using CFD (Computational Fluid Dynamics) numerical simulation. The governing equations of fluid motion (Navier-Stokes equations) are solved numerically in the above-described straight diffuser (FIG. 1(a)) and three versions of the spacer geometry (FIG. 1(b)), small, medium and large shown in FIG. 4, all having a 20 mm outlet 5 with a Turbuhaler mouthpiece inlet using CFX-Tascflow (version 2.11, AEA Technology Engineering Software, Ltd.).

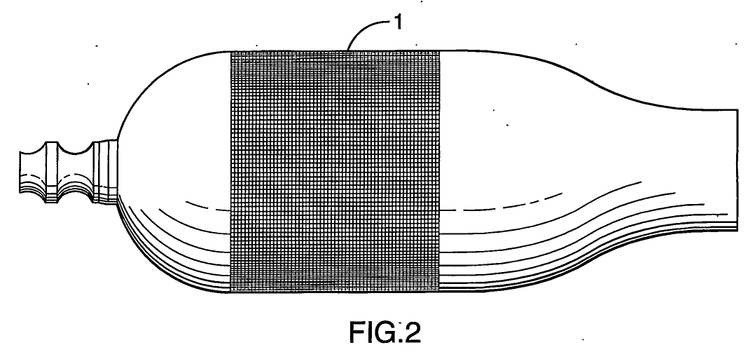

Structured grids having 25 blocks were created as shown in FIG. 2. The grids contain approximately 1,050,000 hexahedric elements, with biased accumulation of nodes towards the wall. See FIG. 2 for a view of the medium size spacer 1 at the middle plane section. Three different grid sizes (1,058,400, 517,880 and 275,400 elements) were tested for the medium spacer 1. Analysis of grid convergence concerning mean velocities and turbulence kinetic energy indicates the size of the adopted grids (around 1,050,000) to be adequate. A modified linear profile scheme that gives second order accuracy in most instances is used in the discretization of the equations and the fluid flow is solved using the standard k-ω turbulence model of Wilcox (1988) with Kato and Launder modification (1993) and near wall treatment for low-Reynolds number (Grotjans and Menter, 1998) (see also documentation of CFX-TASCflow Version 2.11 software, AEA Technology Engineering Software, Ltd.). For the inlet conditions, a steady mean flow rate of 70 L/min, a turbulence intensity of 10% of the mean velocity and a turbulence length scale of 10% of the inlet diameter are used. Doubling or halving these parameters causes little effect on the mean flow quantities (velocity and turbulence kinetic energy) inside the spacers. A zero pressure gauge was applied at the outlet 5. A swirling flow occurs due to the attached non-axisymmetric Turbuhaler mouthpiece. This mouthpiece consists of a double-helical structure (two internal guide walls) rotating 300° over 13.5 mm of length.

A single inhalation cycle following a step breathing function with 2.5 L of air volume and 70.0 L/min of mass flow rate (which is a typical in vivo flow rate with the Turbuhaler) is produced by an in-house breathing machine. During this cycle, aerosol with one dosage of actual drug (terbutaline sulfate, 500 μg), is generated at an intact Turbuhaler and flows through the spacer 1, the idealized mouth-throat cast 4 and the filter 11 (see FIG. 3(a)). After the experiment, the add-on spacer device 1, the idealized mouth-throat cast 4 and filter 11 are washed separately with solvents (typically 15 mL) and aerosol deposition is obtained from UV spectroscopy (Hewlett Packard, model 8452A) concentration measurements of terbutaline sulfate in the solutions obtained from the washings. The total aerosol deposition or deposition efficiency is given by:

$$T . Dep . ( \% ) = \frac{{m}_{spacer} + {m}_{mouth - throat}}{{m}_{spacer} + {m}_{mouth - throat} + {m}_{filter}} \times 100 , \qquad ( 1 )$$

where m_{spacer}, mₘₒᵤₜₕ₋ₜₕᵣₒₐₜ and m_{filter} are the masses of particles deposited on the spacer 1, the idealized mouth-throat cast 4 and the filters 11, respectively.

The total aerosol delivery through the spacer 1 and cast 4 can be calculated by:

$$\begin{matrix}T . Del . ( \% ) = 100 - T . Dep . ( \% ) , \\ or & ( 2 ) \\ T . Del . ( \% ) = \frac{{m}_{filter}}{{m}_{spacer} + {m}_{mouth - throat} + {m}_{filter}} \times 100 , & ( 3 )\end{matrix}$$

which gives the total percentage of medication from the DPI dose that would pass through the mouth-throat region of a patient and would be delivered into the lungs.

Three sets of experiments are performed as follows: 1) Turbuhaler device without add-on devices, 2) Turbuhaler device with a straight diffuser 3 and 3) Turbuhaler® device with spacer 1. The idealized mouth-throat geometry (Stapleton et al., 2000) used for the cast 4 is an average geometrical model for adults based on information available in the literature, supplemented with separate measurements using computed tomography (CT) scans of patients (n=10) with no visible airway abnormalities and by the observation of living subjects (n=5). Experiments on the deposition of aerosols in casts of this mouth-throat geometry indicate that this idealized mouth-throat geometry duplicates average filtering efficiencies in vivo. Both the idealized mouth-throat geometry and the add-on devices were built using Computed Aided Design (CAD) along with a rapid prototyping machine (stereolithography, model FDM 8000, Stratasys, Eden Prairie, Minn.), which produces 3D copies of solid models in acrylonitrile-butadiene-styrene (ABS) plastic. The casts 4 are coated with fluorocarbon FC-725 (3M, St. Paul, Minn.).

In order to eliminate the effect of polydispersity (distribution in particle diameter) on deposition and study the spacer efficiency in terms of the simplified inertial parameter, ρₚ dₚ²Q, monodisperse aerosol deposition tests using the spacer 1 are also performed here. The inhalation flow rate of air is set constant by using a vacuum pump 18, a control valve and a flowmeter 17 (see FIG. 3(b)). Monodisperse aerosols (with dl-α tocopheryl acetate droplets) were generated using a vibrating orifice aerosol generator 12 (TSI Model 345001, St. Paul, Minn.). The aerosol residual charge was neutralized using a radioactive charge neutralizer 13 (TSI Model 3454, St. Paul, Minn.). Dl-α tocopheryl acetate was used because it is a non-volatile liquid at room conditions, detectable in HPLC spectroscopy (also visible to UV spectroscopy (9)), inert to plastic surfaces, and soluble in a large number of solvents including methanol, isopropyl alcohol and heptane. The monodisperse aerosol is directed to an air dilution/mixing chamber 14, where samples are taken to an Aerosizer sampler 15 (TSI, model Mach II, St. Paul, Minn.) to monitor droplet size and aerosol monodispersity throughout the experiment. In order to keep aerosol monodispersity and assuming little effect on the outlet flow field, only the Turbuhaler mouthpiece section 16 (which includes the spiral channels) is used here (instead of the whole inhalation device). The monodisperse aerosol passes through the Turbuhaler mouthpiece section 16, goes through the spacer 1 or straight diffuser 3 and through an idealized mouth-throat geometry 4 before reaching the filters 11. The duration of the experiments is approximately from 1 to 2 hours, depending on inhalation mass flow rate in order to reach good detectable levels. After the experiments, the spacer 1 or straight diffuser 3, the idealized mouth-throat geometry 4 and filter 11 are washed separately with solvents (typically 15 mL) and aerosol deposition is obtained from HPLC (Varian Analytical Instruments, model Pro Star, Walnut Creek, Calif.) concentration measurements of dl-α tocopheryl acetate in the solutions obtained from the washings.

Three different simplified inertial parameters are used (approximately, ρₚ dₚ²Q=6,635, 14,700 and 23,430) for two sets of experiments with and without the spacer 1. The simplified inertial parameters are obtained from a combination of different inhalation flow rates (30, 60 and 90 L/min) and droplet sizes ranging from 2.6 to 5.7 μm diameter.

Statistical analysis of the experimental data (aerosol delivery) for different add-on devices is performed using ANOVA (Analysis Of Variance between groups) for multiple groups and student's “t” test when two groups are compared. The number of experimental repeats are 5 and 3 for Turbuhaler and monodisperse aerosol, respectively. Differences in the experimental results are considered to be statistically significant when P<0.05.

A comparison of the CFD results for the straight diffuser 3 and the three different spacer geometries (small, medium and large) is shown in FIG. 4. The left column of FIG. 4 shows the magnitude of mean velocities and the right column shows turbulence kinetic energies, k. The flow direction is from the left to the right of FIG. 4. The straight diffuser 3, seen on the top, shows that although mean velocities are decreased without flow separation, high levels of turbulence are generated short after the mouthpiece and will convect particles towards the wall by means of turbulent diffusion. In the short and small device, second line from the top, the non-axisymmetrical swirling jet is dissipated in the buffer region 6 and turbulence intensities are much lower, when compared to the straight diffuser 3 case. Turbulence intensities generated after the inlet 9 are slightly advected towards the spacer outlet 5 and potentially passing into the mouth cavity, meaning more particle dispersion and eventually particle deposition. In the medium and the large devices, the jets are also successfully dissipated in the buffer region 6 and both spacer 1 devices have low turbulence intensities at the outlet 5, meaning effective design. Flow separation is seen in the buffer region 6 of the large device, causing flow re-circulation, which may cause particle deposition. The medium sized device has similar performance to the large device and is adopted here due to ergonomic reasons. Large spacers would give difficulties in handling and portability. Note that further detailed optimization can be pursued but the ranges of dimensions or inner/outer envelopes noted above represent the preferred ranges for optimal performance of the spacer 1.

The Turbuhaler aerosol deposition results are shown in FIG. 5. Results are plotted for the case of the inhaler 2 alone, the inhaler 2 plus simplified straight diffuser 3, and inhaler 2 plus spacer 1, each connected to the mouth-throat cast 4. The inhalation flow rate is Q=70 L/min (MMAD=2.5 μm). A straight diffuser 3 shows no improvement since it has a performance of 38.5±2.4% in aerosol delivery (see “Filter” in FIG. 5), similar to the case with no add-on device (38±2.4%). Although the straight diffuser 3 reduces aerosol deposition in the mouth-throat region 4, the deposition in this add-on device 3 itself is high, giving ineffective improvements in overall delivery. The total aerosol delivery through the cast is approximately 55.5±3.3% when the spacer 1 according to the invention is used, indicating an increase of 47% in drug delivery efficiency when compared to the 38±2.4% delivery when the spacer 1 is not used. ANOVA indicates P=0.001.

The monodisperse aerosol delivery results for the experiments with and without the spacer 1 (circle and square marks with fitting curves, respectively) for three different simplified inertial parameters, ρₚ dₚ²Q, are shown in FIG. 6 along with the Stahlhofen et al. (1989) average curve for in-vivo measurements in the mouth-throat (gamma scintigraphy technique). Note that the idealized mouth-throat 4 used here replicates aerosol delivery similarly to the Stahlhofen's average curve (Grgic et al., 2003). The total aerosol delivery was improved by 17, 27 and 107% for s of approximately 6,635, 14,700 and 23,430, and P=0.043, 0.035 and 0.003 from Student's “t” test, respectively. The use of the spacer 1 brought total aerosol delivery to the filter 11 through the mouth-throat region 4 towards the in-vivo measurement curve (Stahlhofen et al., 1989), indicating smooth inlet conditions at the mouth opening and nearly ideal design. Further the spacer 1 geometry succeeds in reducing the amount of deposition in the throat-mouth region thereby avoiding the above mentioned detrimental effects.

Initial design using CFD is shown in this study to be effective in giving overall dimensions of an add-on spacer 1 through analysis of the mean velocity flow field and turbulence intensities without the need for more complex numerical simulations or for lengthy experimental comparisons. The results of aerosol deposition measurements confirm the remarkable improvement in aerosol delivery through a mouth-throat cast 4 predicted through the CFD analysis. The spacer 1 geometry succeeds in giving substantially better aerosol delivery for both Turbuhaler and monodisperse aerosols. The overall dimensions of the proposed add-on spacer 1 are also compact (see FIG. 7 for actual dimensions of the spacer 1 in a picture with Turbuhaler 2), increasing chances of patient's compliance.

The spacer 1 for a commercial dry-powder inhaler 2 is provided. After CFD initial optimization, a geometry having a buffer region 6 to dissipate the jet and a nozzle outlet (approx. 20 mm in diameter) giving relatively low mean velocities and low turbulence intensities is chosen. The performance of the spacer 1 and a simplified straight diffuser 3 are evaluated by measuring the total deposition of actual polydisperse particles and monodisperse aerosol, and consequently the total particle delivery through the cast, in an idealized mouth-throat geometry 4. One inhalation cycle (2.5 L) with flow rate of 70 L/min is used for the polydisperse case. The total delivery of particles with the spacer 1 is increased approximately 47% when compared to experiments without the use of the spacer 1, proving the effectiveness of the proposed design. In the monodisperse aerosol case, improvements for simplified inertial parameters of 6,635, 14,700 and 23,430 were 17%, 27% and 107%, respectively. All increments are statistically significant. The present spacer 1 can be further explored in the development of spacers 1 for different DPIs as well as for design of DPIs themselves.

Although the above description relates to a specific preferred embodiment as presently contemplated by the inventors, it will be understood that the invention in its broad aspect includes mechanical and functional equivalents of the elements described herein.

We claim:

1. A spacer, for disposition between a user's mouth and a medicament inhaler outlet, the spacer comprising a hollow body defining an elongate internal chamber having a longitudinal axis, the spacer having:
a diffuser portion having a spacer inlet adapted to engage the inhaler outlet in communication with the internal chamber, the diffuser portion extending axially outwardly from the spacer inlet;
a buffer portion extending axially from the diffuser portion; and
a nozzle portion having a spacer outlet adapted to engage the user's mouth in communication with the internal chamber, the nozzle portion extending axially inwardly from the buffer portion.

2. A spacer according to claim 1 wherein the diffuser portion is hemi-spherical.

3. A spacer according to claim 1 wherein the buffer portion is cylindrical.

4. A spacer according to claim 1 wherein the nozzle has an internal surface of revolution having an ogee curvature.

5. A spacer according to claim 1 wherein the spacer inlet has a dimension in the range from 7.5 to 12.5 mm.

6. A spacer according to claim 5 wherein the spacer inlet has a dimension of 10 mm.

7. A spacer according to claim 2 wherein the hemi-spherical diffuser portion has a radius in the range from 15 to 25 mm.

8. A spacer according to claim 7 wherein the hemi-spherical diffuser portion has a radius of 20 mm.

9. A spacer according to claim 3 wherein the buffer portion has a radius in the range from 15 to 25 mm.

10. A spacer according to claim 9 wherein the buffer portion has a radius of 20 mm.

11. A spacer according to claim 3 wherein the buffer portion has an axial length in the range from 30 to 50 mm.

12. A spacer according to claim 11 wherein the buffer portion has an axial length of 40 mm.

13. A spacer according to claim 4 wherein the nozzle portion has an upstream inlet radius in the range of 15-25 mm and an downstream diameter in the range of 15-25 mm.

14. A spacer according to claim 13 wherein the nozzle portion has an upstream radius of 20 mm and a downstream diameter of 20 mm.

15. A spacer according to claim 13 wherein the nozzle portion has an axial length in the range of 37.5 to 62.5 mm.

16. A spacer according to claim 15 wherein the nozzle portion has an axial length of 50 mm.

17. A method of optimizing the geometry of a proposed spacer, for disposition between a user's mouth and an outlet of a medicament inhaler, the proposed spacer comprising a hollow body defining an elongate internal chamber, the method comprising:
evaluating the performance of the proposed spacer by measuring the total deposition of particles by:
passing a gas-particle mixture through a test rig with components comprising: the proposed spacer; a mouth-throat model; and a filter;
separately washing each of the test rig components with a solvent to acquire a separate solvent-particle aliquot for each component;
analysing the aliquots to determine a proportion of particles retained in each component relative to a total of particles retained by all components combined; and
comparing the proportion of particles retained by the proposed spacer relative to different spacers of different interior chamber geometry to acquire a measure of the relative efficiency of the proposed spacer when used with said medicament inhaler.

18. A method according to claim 17 wherein a plurality of gas-particle mixtures are passed through the test rig and compared, wherein an inertial parameter of each said gas-particle mixture differs from an inertial parameter of the other gas-particle mixtures, said inertial parameter consisting of ρₚ dₚ²Q/18 μL, where ρₚ is a particle density, dₚ is a particle diameter, Q is an inhalation flow rate, μ is the viscosity of gas and L is the characteristic length scale of the fluid flow path.

19. A method according to claim 17 including in advance of the evaluating step, the step of:
performing computational fluid dynamics numerical simulation to predict the performance of the proposed spacer with internal chamber geometry; and
selecting an internal chamber geometry resulting in relatively low mean velocities and low turbulence intensities at a spacer outlet.

20. A method according to claim 17 wherein the gas-particle mixture comprises a monodisperse aerosol.
